(19) United States
(12) Patent Application Publication
(10) Pub. No.: US 2005/0049707 A1
(43) Pub. Date: Mar. 3, 2005

(54) Cemented artificial disc replacements

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/652,842

(22) Filed: Aug. 29, 2003

(51) Int. Cl. A61F 2/44

(52) U.S. Cl. 623/17.13; 623/908

(57) ABSTRACT

Cementation in place of, or in conjunction with, artificial disc replacements (ADRs) to provide numerous advantages over existing techniques. Methods, instruments, and systems are disclosed. A preferred method of fixing an artificial disc replacement (ADR) to a vertebral endplate according to the invention includes locating a component of the ADR in spaced-apart relation to the vertebral endplate and introducing cement between the component and the vertebral endplate. The “introduction” may be carried out through manual packing or forced injection using inventive instrumentation. The component of the ADR may be a rigid endplate, or it may be constructed of polyethylene or other suitable polymeric material. The component may further articulate with a second component. A system according to the invention involving an artificial disc replacement (ADR) configured for placement between opposing vertebral endplates would comprise a component forming a cavity between the component and one of the vertebral endplates, and a path to fill the cavity with cement. Again, the path may be formed in the component with a channel or groove, with or without a peripheral rim, or the path may be formed through a vertebral body. The system may further include various instruments, including an instrument for urging the component against the vertebral endplate until the cement cures, for injecting the cement prior to insertion of the component, for pressurizing the cement following introduction, or for removing excess, cured cement prior to placement of the ADR.

### FIELD OF THE INVENTION

This invention relates generally to artificial disc replacements (ADRs) and, in particular, to cemented ADRs and instrumentation for use in conjunction therewith.

### BACKGROUND OF THE INVENTION

Current artificial disc replacements (ADRs) are not cemented to the vertebrae. Instead, they rely on the ingrowth of bone or fibrous tissue to stabilize the implant, which is press-fit into place. Press fitting, however, can lead to dislocation and other problems.

U.S. Pat. No. 5,171,281 mentions, in passing, the potential use of cementing agents such as polymethyl methylacrylate “bone cement” for attachment of endplates to adjacent bony vertebral bodies. Rather, the point of novelty resides in disc spacers that possess mechanical properties akin to those of a normal disc to preserve normal functions of the spinal motion segment. A spacer is used having a central core of a soft, biocompatible elastomer shaped and sized so as to approximate the shape and size of a nucleus pulposus of a natural intervertebral disc, with an outer ring of stiffer elastomeric material surrounding the central core to approximate the size and shape of a natural annulus fibrosus. The endplates are comprised of a suitably stiff biocompatible material and affixed, one to each end, to the outer ring/central core. The endplates may also incorporate a mechanism for attachment to adjacent bony vertebral bodies. Such mechanisms include, but are not limited to, mechanical interlock, frictional fit, ingrowth into a porous structure such as a porous sintered surface, hydroxyapatite coatings or cementing agents such as polymethyl methylacrylate “bone cement.” Since this patent refers to the use of cementing agents such as polymethyl methylacrylate as part of a list without any specific details, the need remains for specific guidelines as to the use of cementation in artificial disc (ADR) replacements.

### SUMMARY OF THE INVENTION

Broadly, this invention uses cementation in place of, or in conjunction with, press-fit artificial disc replacements (ADRs). Such an approach offers numerous advantages over existing techniques.

Methods, instruments, and systems are disclosed. A preferred method of fixing an artificial disc replacement (ADR) to a vertebral endplate according to the invention includes locating a component of the ADR in spaced-apart relation to the vertebral endplate and introducing cement between the component and the vertebral endplate. The “introduction” may be carried out through manual packing or forced injection using inventive instrumentation.

The component of the ADR may be a rigid endplate, or it may be constructed of polyethylene or other suitable polymeric material.

The component may further articulate with a second component, which may be substantially rigid and composed of a biocompatible alloy such as titanium or chrome cobalt, or a ceramic may be used. The cement may include an antibiotic. The method may further include the step of preparing a vertebra with hypotensive anesthesia prior to fixing the ADR or preparing a vertebra with a hemostatic agent prior to fixation. A cement-receiving hole or cavity may be formed in a vertebral body, and fluoroscopy may be used, particularly for the cement placement step.

The component of the ADR may include one or more channels to direct the cement between the component and the vertebral endplate. For example, the component may include one or more grooves to direct the cement between the component and the vertebral endplate, or a rim that bears against the vertebral endplate, thereby forming a cavity to receive the injected cement. Additionally, the method may include the steps of forming a passage through the vertebra having the endplate, and introducing the cement through the passage. In all cases, the component may be urged against the vertebral endplate until the cement cures.

A system according to the invention involving an artificial disc replacement (ADR) configured for placement between opposing vertebral endplates would comprise a component forming a cavity between the component and one of the vertebral endplates, and a path to fill the cavity with cement. Again, the path may be formed in the component with a channel or groove, with or without a peripheral rim, or the path may be formed through a vertebral body. The system may further include various instruments, including an instrument for urging the component against the vertebral endplate until the cement cures, for injecting the cement prior to insertion of the component, for pressurizing the cement following introduction, or for removing excess, cured cement prior to placement of the ADR.

### BRIEF DESCRIPTION OF DRAWINGS

FIG. 1A is a sagittal cross section through the spine and an ADR according to the invention;

FIG. 1B is a view of the anterior aspect of the spine and the ADR drawn in FIG. 1A;

FIG. 1C is a view of the top of the ADR drawn in FIG. 1B showing cement being injected through one of the holes in the ADR endplates;

FIG. 2A is a view of the top of an alternative ADR endplate;

FIG. 2B is a view of the front of the ADR endplate drawn in FIG. 2A;

FIG. 3A is the view of the front of a further alternative ADR endplate;

FIG. 3B is a view of the side of the ADR drawn in FIG. 3A;

FIG. 4 is the view of the side of the spine, an ADR, and an alternative method of injecting the cement, in this case through a hole drilled into the vertebrae;

FIG. 5A is the view of the top of an alternative ADR endplate;

FIG. 5B is a lateral view of the spine and the embodiment of the device draw in FIG. 5A;

FIG. 6 is a lateral view of the spine and a different alternative ADR and method. Cement is placed on the surfaces of the ADR and into holes drilled into the vertebrae;

FIG. 7 is a lateral view of an alternative ADR;

FIG. 8 is a lateral view of the spine and a tool used to pressurize the cement into the vertebrae;

FIG. 9 is a lateral view of the spine and a tool used to apply pressure to the ADR endplates; and

FIG. 10 is the view of the top of a vertebra and a tool used to form a cement mantle for a cemented ADR.

### DETAILED DESCRIPTION OF THE INVENTION

Cemented ADRs according to this invention offers numerous advantages to press-fitting. For one, the cement mantle fits the ADR endplates (EP) perfectly. This precise fit eliminates stress risers that are seen with imperfect fits between ADR EPs and the vertebrae. This enables the use of thinner metal components. Second, cement provides better initial fixation of ADRs than press fitting. Viscous cement can be forced into the cancellous bone of the vertebrae to improve the strength of the bone cement junction.

Third, cement allows surgeons to use thicker, all-polyethylene components. Press fit ADR designs that use polyethylene components place thin polyethylene trays between metal endplates. Problems associated with thin polyethylene components are well documented in the hip and knee replacement literature. In particular, wear and fractures of thin polyethylene components frequently require revision surgery. The high complication rate of revision surgery is well documented.

Fourth, cementation provides a means to attach ADRs whose designs do not allow the use of spikes that can be impacted into the vertebrae above and below the ADR. For example, the ADRs described in U.S. Pat. Nos. 4,759,766 and 5,258,031 do not allow the use of large spikes. Fifth, the fixation provided by cement eliminates the large distraction forces required to insert modular convex components. For example, the ADR of U.S. Pat. No. 5,401,269 could be inserted fully assembled, thus eliminating the distraction required to insert the biconvex component, if the ADR were cemented in place. Sixth, cement eliminates the need for bone to grow into ADR endplates. Failure of bone to grow into the ADR endplates of press fit designs can lead to back pain.

Polymethylmethcrylate (PMMA) or other cement may be used according to the invention. Antibiotics could be added to the cement to reduce the risk of infection. The endplates of the vertebrae could be prepared by milling or cutting the surfaces. Alternatively, holes could be drilled through the endplates of the vertebrae.

Hypotensive anesthesia could be used to decrease bleeding from the vertebral endplates. Hemostatic agents such as Platelet Rich Plasma may also be used to help reduce bleeding from the vertebral endplates. Ideally the bone surface is as dry as possible when the ADR is cemented into position.

Cement could be injected while in a highly viscous state. Alternatively, cement allowed to cure further, could be hand packed into the endplates of the vertebrae. The surfaces of the polyethylene components or endplates that lie against the cement could be machined to improve the strength of the component cement junction. For example, the surfaces of the component could be rough. Fluoroscopy could be used during cement injection to minimize the risk injecting cement into the spinal canal. Barium sulfate, or other radio opaque material, could be added to the cement.

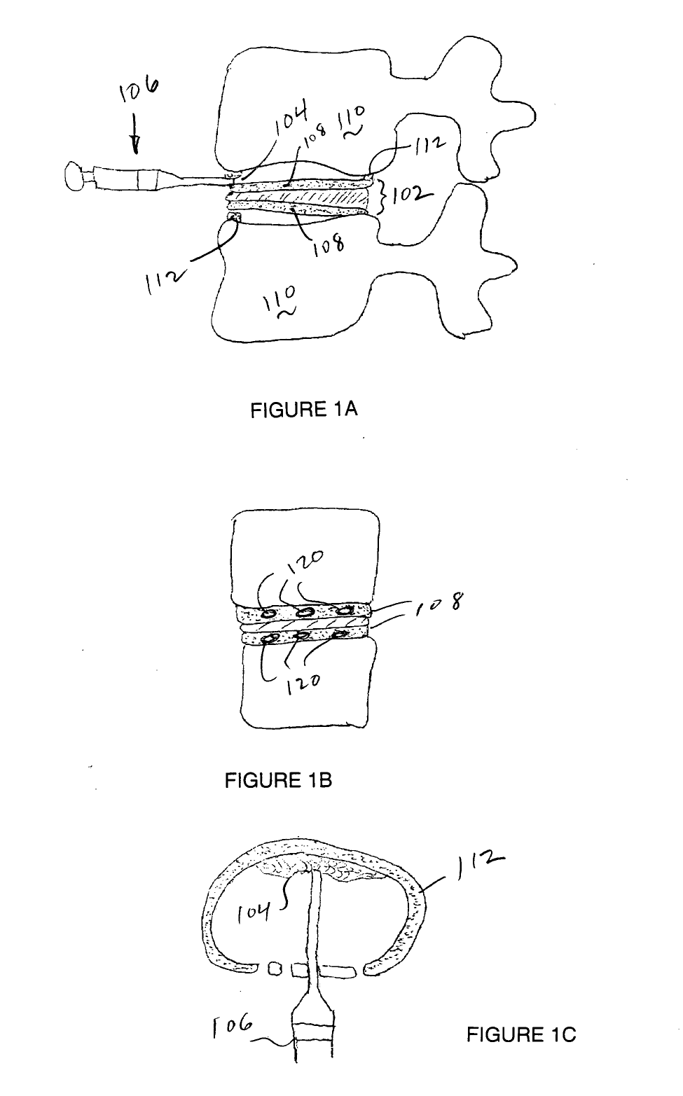

Reference is now made to the drawings, wherein FIG. 1A is a sagittal cross section through the spine and an ADR 102 according to the invention. Cement 104 is injected from a syringe 106 into the space between the ADR endplates 108 and the vertebrae 110. A rigid around the periphery of the ADR endplates 108 creates a space for the cement. A rim 112 further serve to contain the cement.

FIG. 1B is a view of the anterior aspect of the spine and the ADR drawn in FIG. 1A. The holes 120 in the ADR endplates allow the injection of cement and the egress of blood. FIG. 1C is a view of the top of the ADR drawn in FIG. 1B showing cement being injected through one of the holes in the ADR endplates.

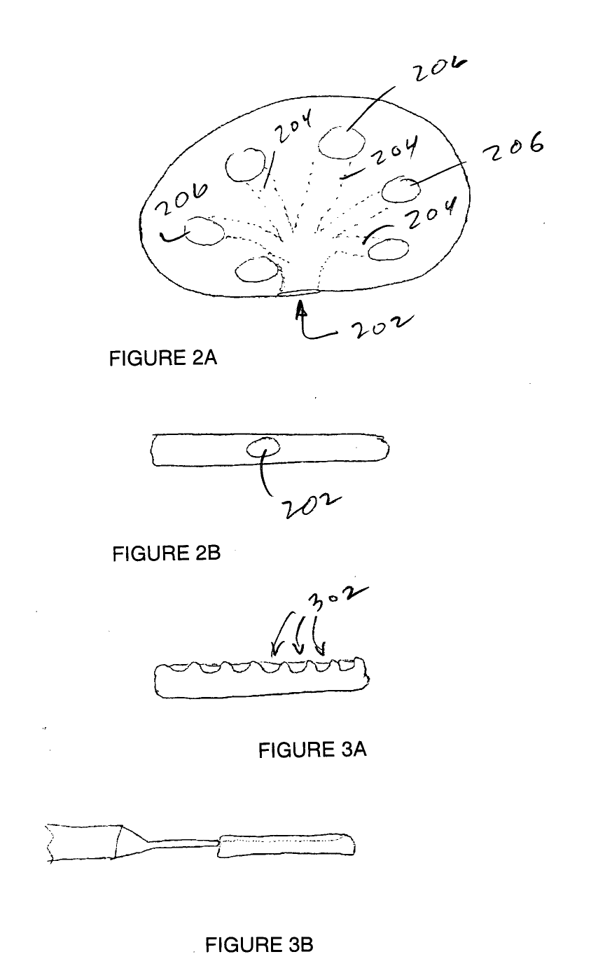

FIG. 2A is a view of the top of an alternative ADR endplate. Cement injected through a hole 202 in the front of the ADR cement travels through channels 204 and is forced out of holes 206 on the side of the ADR endplate that is against the vertebra. FIG. 2B is a view of the front of the ADR endplate drawn in FIG. 2A.

FIG. 3A is the view of the front of a further alternative ADR endplate. Grooves 302 are placed on the surface of the ADR designed to fit against the vertebra. Cement is injected into the grooves before and/or after placement of the ADR. FIG. 3B is a view of the side of the ADR drawn in FIG. 3A. Cement is injected into the grooves of the ADR endplate.

FIG. 4 is the view of the side of the spine, an ADR, and an alternative method of injecting the cement, in this case through a hole 402 drilled into the vertebrae. The cement is injected into a recess 404 formed between the ADR and the prepared vertebral endplate.

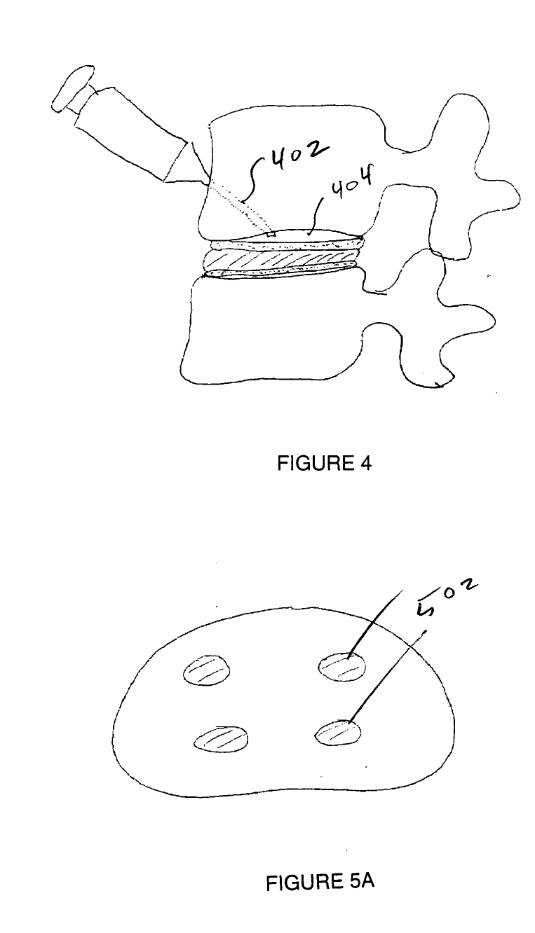

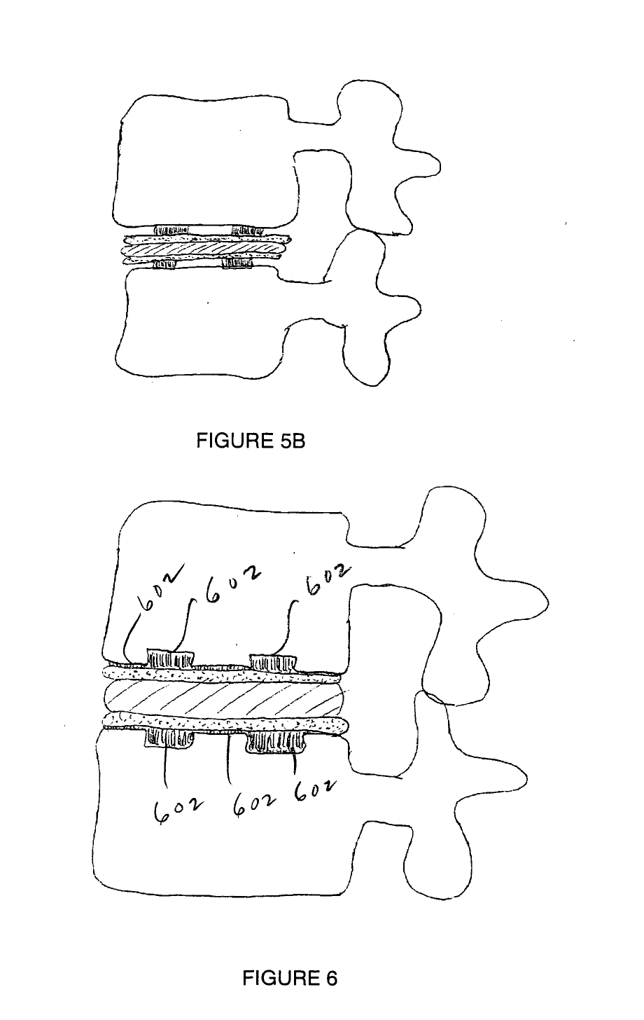

FIG. 5A is the view of the top of an alternative ADR endplate. Cured cement spacers 502 are placed on the surfaces of the ADR endplates that are placed against the vertebrae. Viscous cement is placed around the spacers. The spacers help prevent the uncured cement from scraping off during insertion into the prepared disc space. FIG. 5B is a lateral view of the spine and the embodiment of the device draw in FIG. 5A. The cement spacers are represented by the area of the drawing with vertical lines.

FIG. 6 is a lateral view of the spine and a different alternative ADR and method. Cement is placed on the surfaces of the ADR and into holes drilled into the vertebrae. The cement 602 is represented by vertical lines.

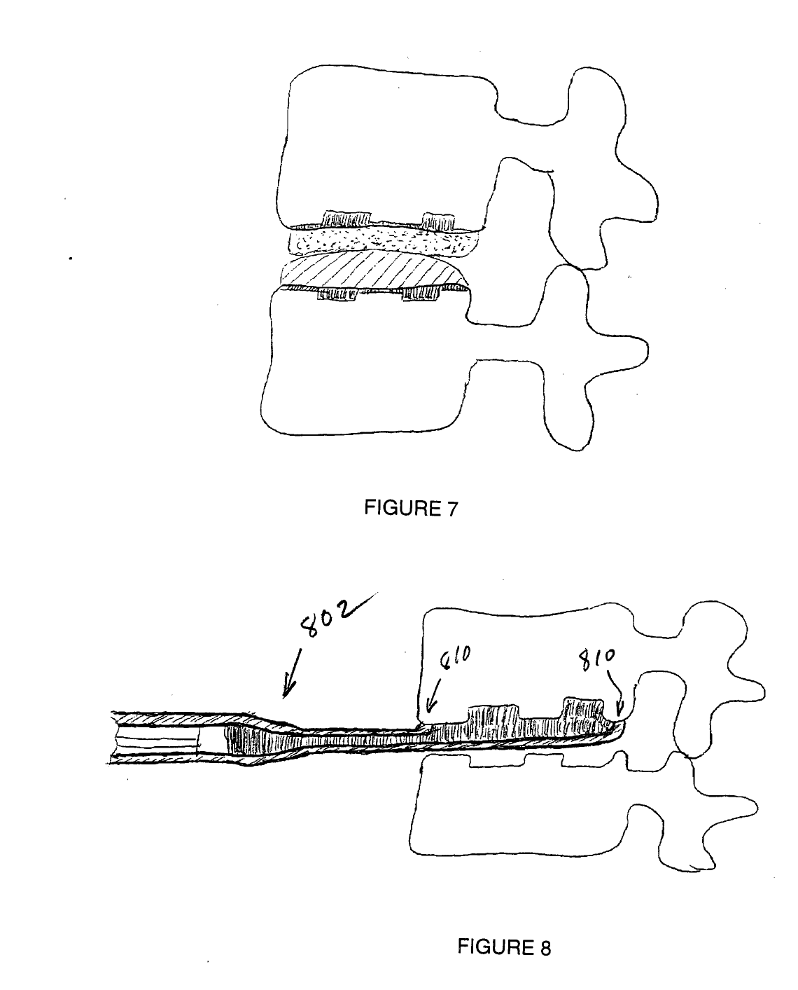

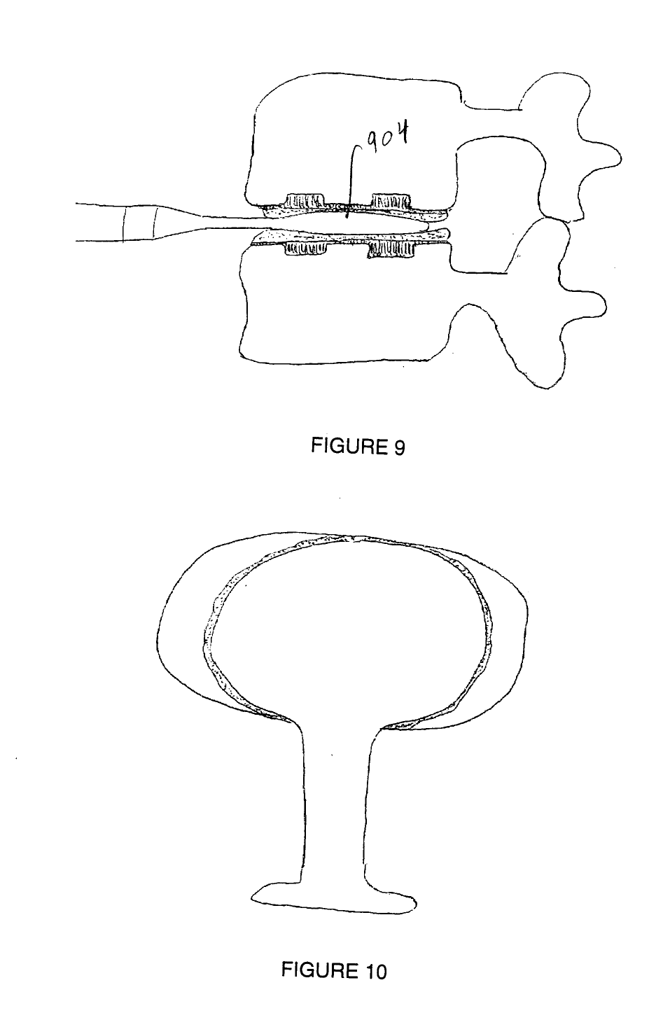

FIG. 7 is a lateral view of an alternative ADR. Note that either endplate can be made completely of polyethylene or other suitable polymeric material. FIG. 8 is a lateral view of the spine and a tool 802 used to pressurize the cement into the vertebrae. The tool 802 has an end that forms a seal 810 against the vertebral endplate. FIG. 9 is a lateral view of the spine and a tool 902 used to apply pressure to the ADR endplates. The tool has an inflatable end 904 that applies pressure to the ADR endplates while the cement cures.

FIG. 10 is the view of the top of a vertebra and a tool used to form a cement mantle for a cemented ADR. The end of the tool that is shaped like the ADR endplate is cemented to the vertebral endplate. The tool is removed after the cement hardens. Removal of the tool allows the surgeon to remove excess cement. It is particularly important to remove cement that protrudes into the annulus fibrosis and into the spinal canal. The ADR is inserted onto the mantle of cement after removing the excess cement. Surgeons may add a thin layer of uncured cement to the ADR prior to placing the ADR endplates onto the cement mantle.

1. A method of fixing an artificial disc replacement (ADR) to a vertebral endplate, comprising the steps of:
locating a component of the ADR within an intervertebral disc space in spaced-apart relation to the vertebral endplate; and
introducing cement between the component and the vertebral endplate.

2. The method of claim 1, wherein the component of the ADR is a rigid endplate.

3. The method of claim 1, wherein the component of the ADR is polyethylene or other suitable polymeric material.

4. The method of claim 3, wherein the component articulates with a second component.

5. The method of claim 4, wherein the second component is substantially rigid.

6. The method of claim 5, wherein the substantially rigid component is constructed of titanium, chrome cobalt, or ceramic.

7. The method of claim 1, wherein the cement includes an antibiotic.

8. The method of claim 1, further including the step of preparing a vertebra with hypotensive anesthesia prior to fixing the ADR.

9. The method of claim 1, further including the step of preparing a vertebra with a hemostatic agent prior to fixing the ADR.

10. The method of claim 1, further including the use of fluoroscopy during the step of cementing the ADR.

11. The method of claim 1, further including the step of forming a cement-receiving hole or cavity in a vertebral body.

12. The method of claim 1, wherein the component of the ADR includes one or more channels to direct the cement between the component and the vertebral endplate.

13. The method of claim 1, wherein the component of the ADR includes one or more grooves to direct the cement between the component and the vertebral endplate.

14. The method of claim 1, wherein the component of the ADR includes a rim that bears against the vertebral endplate, thereby forming a cavity to receive the injected cement.

15. The method of claim 1, further including the steps of:
forming a passage through the vertebra having the endplate; and
injecting the cement through the passage.

16. The method of claim 1, further including the step of pressing the component against the vertebral endplate until the cement cures.

17. The method of claim 1, wherein the step of introducing cement between the component and the vertebral endplate occurs before the endplate is placed in spaced-apart relation to the vertebral endplate.

18. The method of claim 17, wherein the step of introducing cement between the component and the vertebral endplate utilizes a separate tool that is removed before the endplate is placed in spaced-apart relation to the vertebral endplate.

19. The method of claim 1, further including the step of shaping the vertebral endplate before introducing the cement.

20. A system including an artificial disc replacement (ADR) configured for placement within a vertebral disc space between opposing vertebral endplates, the ADR comprising:
a component forming a cavity between the component and one of the vertebral endplates; and
a path to fill the cavity with cement.

21. The system of claim 20, wherein the path is formed in the component.

22. The system of claim 20, wherein the path is a channel or groove.

23. The system of claim 20, wherein the component includes a peripheral rim to form the cavity.

24. The system of claim 20, wherein the component is a rigid endplate.

25. The system of claim 20, wherein the component is polyethylene or other suitable polymeric material.

26. The system of claim 20, wherein the component is a rigid endplate.

27. The system of claim 20, further including an instrument for urging the component against the vertebral endplate until the cement cures.

28. The system of claim 20, further including an instrument for injecting the cement prior to insertion of the component.

29. The system of claim 20, further including an instrument for pressurizing the cement following introduction.

30. The system of claim 20, further including an instrument for removing excess, cured cement prior to placement of the ADR.

31. The system of claim 20, further including two components, one proximate to each of the opposing vertebral endplates; and
paths for injecting cement between each component and its respective vertebral endplate.

32. The system of claim 20, wherein the component includes one or more protuberances to create a space for the cement.

33. The system of claim 20, wherein the protuberances are PMMA spacers.
